(19) United States
(12) Patent Application Publication
(10) Pub. No.: US 2019/0008746 A1
(43) Pub. Date: Jan. 10, 2019

(54) Oral Hygiene Products and Method of Using the Same

(71) Applicant: Dental Research, Inc., Huntington, NY (US)

(72) Inventor: Charles Leonhardt, Huntington, NY (US)

(21) Appl. No.: 16/114,551

(22) Filed: Aug. 28, 2018

Related U.S. Application Data
(63) Continuation-in-part of application No. 15/225,630, filed on Aug. 1, 2016, now Pat. No. 10,058,497.
Application No. 15/225,630 is a continuation of application No. 14/831,598, filed on Aug. 20, 2015, now Pat. No. 9,427,389.
Application No. 14/831,598 is a continuation of application No. 14/168,564, filed on Jan. 30, 2014, now Pat. No. 9,138,397.
Application No. 14/168,564 is a continuation of application No. 13/385,420, filed on Feb. 17, 2012, now Pat. No. 8,679,463.
Application No. 13/385,420 is a continuation of application No. 11/365,167, filed on Mar. 1, 2006, now Pat. No. 8,163,271.

(51) Int. Cl. A61K 8/67; A61K 8/49; A61K 31/375; A61K 8/29; A61K 8/60; A61K 8/02; A61K 8/25; A61K 8/22; A61K 8/34; A61K 8/21; A61K 8/24; A61K 8/365; A61K 8/368; A61K 33/42; A61Q 11/00

(52) U.S. Cl. CPC A61K 8/676; A61K 8/4973; A61K 2300/00; A61K 2800/43; A61K 8/29; A61K 8/60; A61K 8/0204; A61K 8/602; A61K 8/25; A61K 8/022; A61K 8/22; A61K 8/345; A61K 2800/59; A61K 8/21; A61K 8/24; A61K 8/34; A61K 2800/92; A61K 8/365; A61K 8/368; A61K 8/49; A61K 8/4926; A61K 2800/30; A61K 33/42; A61Q 11/00; A61K 31/375

(57) ABSTRACT

A composition for highly efficient and quick plaque and tartar removal and inhibition including pure ascorbic acid crystals and/or granulars (vitamin C) and an enamel repairing composition such as hydroxyapatite. The composition can contain from about 10% up to 90% of ascorbic acid without compromising the integrity of tooth enamel because hydroxyapatite will repair any damage to the enamel. The composition can be used as dental powder, toothpaste. prophy paste, mouthwash, mouth spray and chewing gum in order to cover a wide range of consumers in different settings.

### CROSS REFERENCE TO RELATED APPLICATIONS

This application is a continuation-in-part of U.S. patent application Ser. No. 5/225,630, filed on Aug. 1, 2016, now pending, which is a continuation of U.S. patent application Ser. No. 14/831,598 filed on Aug. 20. 2015, now U.S. Pat. No. 9,427,389, which is a continuation of U.S. patent application Ser. No. 14/168,564, filed on Jan. 30, 2014, now U.S. Pat. No. 9,138,397, which is a continuation of U.S. patent application Ser. No. 13/385,420 filed on Feb. 17, 2012, now U.S. Pat. No. 8,679,463, which is a continuation of U.S. patent application Ser. No. 11/365,167 filed on Mar. 1, 2006, now U.S. Pat. No. 8,163,271. The patent applications identified above are incorporated here by reference in their entirety to provide continuity of disclosure.

### FIELD OF THE INVENTION

The invention relates to compositions and methods useful in promoting oral health and hygiene.

### BACKGROUND OF THE INVENTION

Tooth decay and gum inflammation are related to the activity of microbial plaque, which consists of bacterial products, leukocytes, epithelial cells and saliva components. In the presence of saliva, proliferating bacteria attach to places with retained food such as gum line, tongue, tooth spacing, pits and fissures. The bacteria decompose retained food, releasing toxic substances and forming plaque and tartar (an advanced formation of plaque). This results in bad breath, gum inflammation and dental caries.

The majority of oral care products are artificial and harmful when ingested. Some of them induce allergic reactions and others are even carcinogenic when presented in high dosages. Antibiotics have also been used to fight plaque formation. However, antibiotic applications usually result in the development of resistant microorganisms. As a consequence, there is a trend toward the use of safe ingredients in oral care products. Also, the emphasis in oral hygiene has been placed on chemical methods of removing plaque and tartar. These chemical methods eliminate the need for the mechanical action of scrapers.

The present invention relates to a safe oral composition, which chemically removes plaque and tartar. Moreover, it inhibits further plaque accumulation and tartar formation. Thus, the invention is highly efficient in plaque and tartar removal and inhibition. The invention is also a natural teeth whitener and a preservative.

### DESCRIPTION OF RELATED ART

Oral care products containing safe ingredients, such as baking soda, are well known. However, the high concentration of baking soda required to provide adequate cleaning is abrasive and distasteful.

Saponin is another type of safe plaque cleanser, which produces foaming and cleans without the use of artificial surfactants. However, saponins are of plant origin (Quillaja and/or Yucca tree) and have to be extracted from plants, which is a laborious and time-consuming process.

Ascorbic acid (vitamin C) is a safe ingredient and has been used in oral compositions. However, those compositions usually contain only a small amount of ascorbic acid and mainly rely on other cleaning ingredients. Other compositions use ascorbic acid but fail to efficiently utilize its strong and safe cleaning capacity heavily relying on catalysts for auto-oxidation, such as copper, and the synergetic action of other cleaning agents. None of the above prior art describes, suggests or renders obvious the enormous cleaning capacity of pure ascorbic acid crystals and/or granulars unaided by other cleaning agents.

### SUMMARY OF THE INVENTION

Plaque and tartar grows within a short period of time after eating or cleaning, depending on individual saliva chemistry, a biofilm will form on the tooth enamel. (This could occur within an hour or two). Biofilm is an extremely tough substance, and not easily removed. It provides a lattes nesting place for plaque to deposit and grow. Biofilm is a problem even on hulls of ocean vessels where is adheres and then provides barnacles a place to nest and grow. Plaque is a soft white form of bacteria that nests in the lattes of the biofilm. It is relatively easy to remove, if removed within a reasonable time. If not removed it calcifies and becomes tartar. The present invention is designed to attack the biofilm, and remove the calcified plaque (tartar).

Saliva is the first stage of human and animal digestive system. It is a complex liquid that does not the same chemistry in every person's mouth. There are also hundreds of glands in the human mouth that produce saliva in varying degrees and chemistry. The main producers of saliva, but not limited to, are the Sublingual, Submandibular, and Parotid glands. Salvia provides an aid to chewing and swallowing, as well as being the first stage of the digestive system. After meals or snacks it continues to clean mouth, teeth and gums. Without saliva it would be very difficult to swallow foods.

Saliva contains acids, enzymes, buffers, acids, and water among other ingredients. The typical human produces about a quart of saliva in a day. Part of saliva's continuing job is to breakdown, liquefy the remaining food and bacteria that accumulates on, and between teeth, acid gums. It is a never-ending process that takes place in the mouth.

Saliva chemical ingredients vary from person to person, and may not contain certain ingredients in proportion to effectively remove the tough biofilm (lattes, or nesting place) on which bacteria and tartar to grow. A large portion of the world population lacks those bacteria fighting ingredients, resulting in serious illnesses that are not readily associated to oral hygiene deficiencies. Studies over the past ten years have contributed major health disorders to oral hygiene deficiencies such as heart, diabetes, even dementia. Saliva chemistry will largely determine whether a person will have healthy teeth and gums.

Most humans require aid in fighting tooth and gum disorders, and resort to brushing, flossing, and mouth wash, and in cases of weak salvia chemistry; scrapings several times per year by a professional orthodontist.

The present invention has three main active ingredients: ascorbic acid that will dissolve plaque and tartar and attack biofilm, and hydroxyapatite and sodium fluoride to repair bone, enamel and microscopic fissures thereon.

When the present invention comes into contact with saliva it will instantaneously liquefy and in turn excite salvia glands to accelerate production. This event actually reinforces the present invention by using saliva ingredients that have some bacteria fighting ingredients, although the present invention by itself will remove tartar and plaque, as proven in vivo testing. It works better reinforced by saliva components that facilitate the flow between teeth gums, and any microscopic void, or cavity.

Once the present invention liquefies in the mouth, and reinforced with whatever bacteria fighting components exist in saliva, it will seep into the smallest voids and crevices to attack bacteria in whatever form it is in, plaque, tartar, or biofilm, such as in the case of gingivitis.

Simultaneously with removal of plaque and tartar, the hydroxyapatite and sodium fluoride component will also liquefy and seep into any voids in bone, enamel and tooth structure.

It is extremely important to prevent plaque and tartar from forming at and below the gum line. Both plaque and tartar are irritants to gum tissue and if not removed will cause separation between gum and tooth enamel, if left untreated will cause gingivitis or other forms of gum infection, as well as bone deterioration. Gum tissue will not tighten around teeth in the presence of plaque and tartar. Gum tissue will also become infected when in contact with tartar. If not corrected gums will recede and separate from the teeth allowing bone to deteriorate.

In severe cases of gingivitis, the gingiva may also become infected. The present invention will restore its integrity and allow it to properly bond with bone gum tissue and teeth. As plaque and tartar are removed the healing and rebuilding process will quickly evolve. It has been surprisingly observed that once tartar is removed the problem of receding gum line is greatly reduced and eliminated. In some cases, the present invention promoted gum growth. There are surprising reports that in some severe cases of gingivitis where deterioration of bone occurred, after prolonged use of the present invention, the bone rejuvenated and showed regrowth.

The present invention also contain fluoride in quantities that help fight cavities all of the ingredients are safe and ingestible as opposed to other oral hygiene products. There are no or nearly zero abrasives, or carcinogenic ingredients.

While the prior art avoids using high concentrations of ascorbic acid as harmful to the tooth enamel, the preferred embodiment of the present invention can contain from about 10 up to 90% of ascorbic acid since hydroxyapatite will efficiently repair and protect the enamel. Also, while the prior art compositions require treatment longer than one minute, the preferred embodiment of the present invention requires only a 30 second treatment.

Again, the present invention is directed to an oral composition for plaque and tartar removal and control. The composition contains pure crystals and/or granular of ascorbic acid (vitamin C) and hydroxyapatite. When used in high concentrations (from 10% up to about 90%), ascorbic acid is highly efficient in quickly dissolving and detaching plaque and tartar upon contact. Such high concentrations will not compromise the integrity of the tooth enamel because hydroxyapatite will repair and protect it. In one exemplary embodiment, any irritation, such as, oral mucosa that may be caused by the ascorbic acid can be prevented by using some form of menthol in composition.

The invention chemically dissolves plaque and tartar in usually less than 60 seconds. It eliminates the need for additional cleaning agents and the mechanical action of scrapers. Furthermore, the invention is highly efficient in inhibiting plaque accumulation and tartar formation if used consistently.

In one embodiment, a plaque and tartar removal and prevention dental prophy paste with enamel strengthening, repairing and protecting qualities for oral hygiene can comprise a high concentration of ascorbic acid for plaque and tartar removal, at least 20% ascorbic acid; a blend of hydroxyapatite crystals and sodium fluoride sufficient to strengthen tooth enamel, protect the tooth enamel against acid damage and repair fissures in the enamel in which plaque causing bacteria collect, the blend containing between 0.2-0.5% hydroxyapatite crystals and about 0.05-0.5% sodium fluoride; an amount of zinc citrate trihydrate sufficient to prevent plaque adherence to the tooth enamel; and an amount of anhydrous glycerin. In use, when combining a sufficient quantity of the plaque and tartar removal and prevention prophy paste with saliva in a mouth forms a liquid that chemically dissolves and detaches plaque from teeth, gum and tongue and quickly breaks a barrier between the teeth and calcified tartar.

In some embodiments, the prophy paste can contain one or more of the following: an amount of tricalcium phosphate to use as a carrier for the ascorbic acid. the amount being between 0.25-2.5% tricalcium phosphate; an amount of hydrated silica sufficient to use as a whitener, the amount being between 0.25-4.0% hydrated silica; an amount of sodium saccharine sufficient to use as a sweetener, the amount being between 0.001-0.2% sodium saccharine; an amount of sodium benzoate, the amount being between 0.05-0.4% sodium benzoate; an amount of spearmint/peppermint flavor, the amount being between 0.05-0.4% spearmint/peppermint flavor; an amount of calcium orthophates, the amount being between 0.25-2.5% calcium orthophates; an amount of tricalcium phosphate, the amount being between 2.0-7.5% tricalcuim phosphate; and an amount of sodium lauryl sulfate, the amount being between 0.25-2.5% sodium lauryl sulfate.

### DETAILED DESCRIPTION OF THE INVENTION

It is an object of the present invention to provide improved plaque and tartar removal and control. The invention is based on the enormous cleaning capacity of pure ascorbic acid crystals, granulars and/or any other form of pure ascorbic acid unaided by additional cleaning ingredients. Please note, a granular form of ascorbic acid may be used instead of the crystalline form.

The present invention uses high concentrations of ascorbic acid where hydroxyapatite is used to repair and protect the tooth enamel. It is worthy to note that ascorbic acid is the preferred compound of the present invention but any acidic compound that incorporates high cleaning of may be used as a substitute for the ascorbic acid.

Ascorbic acid (vitamin C) is a non-toxic compound which is harmless when ingested. It has been known that vitamin C deficiency causes scurvy when collagen formation is compromised. In addition, a diet deficient in vitamin C renders gingiva more susceptible to bacterial attacks. Thus, ascorbic acid is not only harmless but also important for the human organism's healthy functioning. Therefore, it is one of the objects of the present invention to provide a safe oral hygiene composition, which promotes human health.

The harmless nature of the present invention makes it even more suitable for use by children. Since many parents are not able to effectively control their children's oral hygiene, the present invention solves this problem by providing a safe, healthy and efficient cleaning procedure, eliminating the need for long and extensive brushing.

Different studies have confirmed the role of microbial plaque as a major factor in dental caries and periodontal diseases. The most common types of periodontal disease are gingivitis and periodontitis. Gingivitis is an early stage gum disease characterized by gum inflammation, swelling and bleeding. Periodontitis is a late stage gum disease, in which tooth supporting bone is slowly lost. In view of plaque's major role in dental diseases, one of the objects of the present invention is to provide a safe and effective oral hygiene composition for combating bacteria associated with dental plaque, caries, and periodontal diseases.

Pure ascorbic acid crystals and/or granulars (more than 70% purity) in the form of dental powder can be viewed as the most efficient embodiment of the invention. The size of the crystals and/or granulars can be but is not limited to 5 u-50 u. When used in high concentrations (ranging from over 10% to about 90%), the ascorbic acid crystals and/or granulars have enormous cleaning capacity, eliminating the need for other cleaning agents and further mechanical cleaning. Such high concentrations are extremely effective in killing a wide spectrum of bacteria comprising the oral microflora: Actinomyces viscosus, alpha Streptococcus, Candida albicans, Escherichia coli, Pseudomonas aeruginosa, Staphylococcus epidermidis and Streptococcus mutants.

The present invention does not compromise the integrity of the tooth enamel even in the presence of 90% or more of ascorbic acid since the hydroxyapatite and sodium fluoride will protect and repair the enamel. The invention can contain from 0.2% up to about 1.0% of hydroxyapatite but the preferred amount is 0.5% by weight and about 0.05% up to about 1.0% sodium fluoride but the preferred amount is 0.24% by weight.

Hydroxyapatite is the major component, and an essential ingredient, of normal bone and teeth. It is a thermally unstable compound, decomposing at temperatures from about 800-1200° C., depending on its stoichioinetry. Hydroxyapatite supports bone ingrowth and osseointegration when used in orthopaedic, dental and maxillofacial applications.

Hydroxyapatite adheres to the surfaces of teeth and promotes their recalcification and strengthening. It has been successfully used in a dental fine filling method for protecting and restoring pits, fissures and lesions in enamel. Also, hydroxyapatite absorbs dental plaque, which causes tooth decay. Studies have showed the absorption of oral Streptococci by hydroxyapatite.

In addition to ascorbic acid and hydroxyapatite, the dental powder form of the present invention contains about 10-20% tricalcium phosphate, 0.1-0.2% menthol and 4-6% zinc citrate.

Tricalcium phosphate is used as a filler and carrier for the ascorbic acid crystals, menthol is a local anesthetic and counterirritant, and zinc citrate is an agent preventing plaque adherence to the tooth enamel. The dental powder can also contain fluoride (especially in regions where coater has an insufficient amount of fluoride), sweeteners, flavoring and coloring agents.

Ascorbic acid is also a natural whitener, eliminating the need for additional whitening agents. However, the dental powder can contain additional whitening ingredients such as sodium carbonate peroxide (about 3-5%) and hydrated silica (about 5-7%).

The ascorbic acid crystals and/or granulars can also be included in water-free pastes such as a water-free glycerol paste, chewing gums, aqueous and alcohol aqueous solutions. The aqueous solutions can contain up to 80% water and the alcohol solutions can contain up to 30% denatured alcohol and up to 50% water. The alcohol solutions can contain thymol, menthol or other alcohols.

It is important to note that ascorbic acid is a preservative and its solutions need not be specially stored or packaged in sealed containers. Given the invention's preserving characteristics, its solutions can be made with tap water without compromising the solutions' cleaning properties. However, purified water is preferred. Also, the invention can use additional preservatives such as sodium benzoate in an amount of about 0.2-0.4%.

The present invention can contain, other ingredients including sweeteners, flavoring and coloring agents which could be added to achieve different tastes and flavors. Suitable sweeteners are sodium saccharine, aspartame, cyclamates, sucrose, sorhitol, mannitol, and maltitol. The preferred sweetener, however, is sodium saccharine (0.01-0.02%). Suitable flavoring agents are natural and synthetic oils. The preferred flavoring is a combination of spearmint and peppermint in an amount of about 0.2-0.4%. However, the invention can contain other flavoring agents such as cinnamon oil, wintergreen oil, bay oil, citrus oil, lemon oil, lime oil, clove oil and menthol.

The invention can also contain medications or bioactive ingredients such as antifungal, anti-inflammatory, antibiotic, anti-bacterial, analgesic and immunosuppressive agents.

The dental powder form of the present invention becomes liquid upon contact with saliva and chemically dissolves and detaches plaque from teeth, gum and tongue in about 15 to 90 seconds. Furthermore, the formed liquid quickly breaks the barrier between the teeth and calcified tartar.

Similarly, the solution forms of the invention react with plaque and form a fatty like substance, which detaches and washes plaque and tartar away from teeth, gum and tongue. This eliminates the need for mechanical scrapers, and their maintenance and storage. The solutions can also remove plaque between teeth depending on the strength of the rinse which provides access to the narrowest places. The amount of crystals and/or granulars in those solutions can be varied in order to comfortably achieve the desired taste and effectiveness.

Thus, one of the improved functions of the present invention is fast and efficient plaque removal and inhibition. Plaque is a fatty substance consisting of bacterial colonies surrounded by a gel-like intercellular substance derived chiefly from the bacteria themselves. Plaque also contains saliva, epithelial cells and leukocytes. It usually accumulates on tooth surface, gum, gum line and tongue resulting in had breath, gum disorders and caries. Bacterial colonies of the plaque use dietary carbohydrates as a source of energy producing acids. The acids demineralize tooth enamel and dentine, attacking gum tissue and reacting with the calcium in the teeth.

Persons brushing their teeth normally take approximately one minute, limiting exposure time to either chemical or mechanical action. Most dentists and oral hygienists recommend longer treatments for efficient plaque removal. However, the invention acts quickly to chemically dissolve plaque and tartar, leaving smooth tooth surfaces, clean gum and tongue in less than 15 to 90 seconds. Furthermore, ascorbic acid softens plaque formed between teeth and loose gum. Plaque accumulation is inhibited resulting in a tightened gum line, fresh breath and refreshing mouth taste. As a consequence, dental caries and periodontal diseases are effectively prevented.

Another improved function of the present invention is fast and efficient tartar removal and inhibition. Tartar constitutes an advanced formation of plaque. Tartar is formed when inorganic salts and phosphate in saliva are deposited on plaque. This leads to calcification and a hard surface formation, which is difficult to remove. Leaving tartar for longer time periods may result in serious tooth and gum disorders. Tartar is usually removed by mechanical means such as ultrasonic scrapers, picks and brushes. However, ascorbic acid tends to break down the barrier between the teeth and calcified tartar. Thus, each application of the invention shrinks and inhibits tartar formation, resulting, in the prevention of dental caries and periodontal diseases.

The present invention can be used in a variety of commercial product forms such as mouthwash, mouth spray, toothpaste, dental powder, prophy paste and chewing gum in order to cover a wide range of consumers in different settings. While mouthwash, toothpaste and dental powder are more suitable for domestic use, mouth spray and chewing gum can be universally applied.

The toothpaste and dental powder form of the invention are more suitable for use in domestic settings, where they are applied to teeth with or without a brush. After toothpaste application and brushing is completed, the toothpaste and dental powder are rinsed with water and expectorated. Similarly, the mouthwash form of the present invention can be comfortably used in domestic settings, where a person has the opportunity to extensively rinse his or her oral cavity. After the rinse is completed, the mouthwash is usually expectorated and the oral cavity is rinsed with water. It is important to note that any accidental swallowing will not be harmful given the ascorbic acid's safe nature.

In contrast to the mouthwash and toothpaste forms, which are more suitable for domestic use, the mouth spray and chewing gum forms of the invention can be universally used. Although many people are willing to take oral hygiene measures throughout the day, they find those measures to be inconvenient, and sometimes awkward. For example, they may not have constant access to a bathroom or a sink, feeling unable to use toothpastes and mouthwashes. Also, they may be uncomfortable being seen carrying around items such as a toothbrush, a toothpaste or a bottle of mouthwash. Consequently, there is a great need for oral hygiene products which are convenient, portable and swallowable. Such products are the mouth spray and gum forms of the present invention.

Mouth spray requires spraying the solution into the oral cavity and retaining it for about 15 to 90 seconds to allow efficient plaque and tartar removal. After that the solution could be safely swallowed or simply expectorated. Mouth sprays could be packaged in portable bottles in order to fit pockets, purses and bags.

Chewing the gum form of the present invention is another form of universal application. Plaque and tartar can be dissolved and dislodged by chewing the gum for a short time period sufficient to remove plaque and tartar. Furthermore, a person can continue chewing the gum even after plaque is removed in order to inhibit plaque accumulation and tartar formation for longer time periods.

Prophy paste is a type of teeth cleanser used by dentist or hygienist to polish teeth. These pastes contain abrasives, e.g., pumice stone, quartz, etc., which helps scrub stains and dirt off the enamel of teeth. The propy paste can come in three stages: course, medium, and fine based on the content and size of the abrasives that are added to the basic base carrier. Prophy paste is usually coarser than regular toothpaste and is the perfect finishing touch to the end of a prophylaxis appointment because it makes teeth feel smooth and look polished. While prophy paste cleans and removes stains, if it is used incorrectly it can be harmful to one's teeth as the abrasives can erode the tooth structure. Also, if someone is not trained to clean teeth, polishing with prophy paste can be uncomfortable and is likely to irritate both the gums and teeth. In use, dentists or hygienists apply prophy paste to their patients' teeth using a prophy angle, which is a brush that is in the shape of a rubber cup. The little cup on the end of the prophy angle spins, working to polish the teeth.

The prophy paste of the present invention is a prophy paste that contains no to very little abrasive but with a high amount of ascorbic acid, e.g. 30-75%. This prophy paste breaks down tartar fast and efficiently by allowing the ascorbic acid to break down the barrier between the teeth and calcified tartar. Thus, the application of the prophy paste shrinks and inhibits tartar formation, resulting in the prevention of dental caries and periodontal diseases. The prophy paste can also contain hydroxyapatite crystals and sodium fluoride sufficient to strengthen tooth enamel, protect the tooth enamel against acid damage and repair fissures in the enamel in which plaque causing bacteria collect, e.g. between 0.2-0.5% hydroxyapatite crystals and about 0.05-0.5% sodium fluoride.

The prophy can also contain 0.001-0.2% sodium saccharine; 0.05- 0.4% sodium benzoate; 0.05-0.4% spearmint/peppermint flavor; 0.5- 4.0% zinc citrate trihydrate; 15.0.-40.0% anhydrous glycerin; 0.25-2.5% calcium orthophates; 0.25-4.0% hydrated silica; 2.0-7.5% tricalcuim phosphate; 0.25-2.5% sodium lauryl sulfate: and 0-3.0% other ingredients.

Below are example compositions using the preferred embodiments of the present invention.

### EXAMPLE 1

The first example is a medicated chewing gum containing:
69.0% masticatory gum core;
20.0% ascorbic acid crystals and/or granulars;
0.1% sodium saccharine;
0.5% hydroxyapatite;
0.2% spearmint/peppermint flavor;
0.24% sodium fluoride;
0.001% blue 41;
5.0% zinc citrate trihydrate;
2.0% sucralose;
1.0% polymer coating for sugar-free chiclets;
0.1% sodium benzoate;
1.0% titanium dioxide; and
0.859% other ingredients.

### EXAMPLE 2

The second example is a tooth powder containing:
70.0% ascorbic acid crystals and/or granulars;
12.0% tricalciumphosphate or dicalciumphosphate;
0.1% menthol natural crystalline powder;
0.2% spearmint/peppermint flavor;
0.24% sodium fluoride;
3.0% sodium carbonate peroxide;
5.0% hydrated silica;
5.0% zinc citrate trihydrate;
2.0% sucralose;
0.5% hydroxyapatite;
1.0% titanium dioxide; and
0.96% other ingredients.

### EXAMPLE 3

The third example is an alcohol-free mouthwash as deionized water containing:
25.0% ascorbic acid crystals and/or granulars;
0.01% sodium saccharine;
0.2% spearmint/peppermint flavor;
0.001% blue #1;
5.0% zinc citrate;
0.7% cethylpyridium chloride;
10.0% glycerin:
0.1% polymer 407;
0.5% hydroxyapatite;
58% deionized water; and
0.489% other ingredients.

### EXAMPLE 4

The fourth example is a mouthwash with alcohol and deionized water containing:
20.0% ascorbic acid crystals and/or granulars;
10.0% sorbitol solution;
0.01% sodium saccharine;
0.2% sodium benzoate;
0.2% spearmint/peppermint flavor;
0.0001% blue #1;
5.0% zinc citrate;
0.5% hydroxyapatite;
10.0% glycerin;
0.1% polymer 407;
0.24% sodium fluoride;
0.1% menthol in denatured alcohol;
0.1% thymol in denatured alcohol;
20.0% denatured alcohol with menthol and thymol;
33% deionized water; and
0.5499% other ingredients.

### EXAMPLE 5

The fifth example is a toothpaste containing:
34.0% ascorbic acid crystals and/or granulars;
10.0% sorbitol powder;
0.5% hydroxyapatite crystals;
0.01% sodium saccharine;
0.2% sodium benzoate;
0.2% spearmint/peppermint flavor;
5.0% zinc citrate trihydrate;
30.0% anhydrous glycerin;
0.24% sodium fluoride;
3.0% calcium orthophates;
5.0% hydrated silica;
10.0% tricalcuim phosphate;
1.0% sodium lauryl sulfate; and
0.85% other ingredients.

### EXAMPLE 6

The sixth example is a toothpaste containing:
45.0% ascorbic acid crystals and/or granulars;
8.0% sorbitol powder;
0.5% hydroxyapatite crystals;
0.01% sodium saccharine;
0.2% sodium benzoate:
0.2% spearmint/peppermint flavor:
3.0% zinc citrate trihydrate;
28.0% anhydrous glycerin;
0.24% sodium fluoride;
1.0% calcium orthophates;
4.0% hydrated silica:
8.5% tricalcuim phosphate;
1.0% sodium lauryl sulfate; and
0.35% other ingredients.

### EXAMPLE 7

The seventh example is a toothpaste containing:
40.0% ascorbic acid crystals and/or granulars;
2.85% sorbitol;
0.5% hydroxyapatite;
1.0% peppermint oil;
0.5% lemon oil flavor;
2.0% zinc citrate trihydrate;
38.11% anhydrous glycerin;
0.24% sodium fluoride powder;
6.0% tricalcuim phosphate;
2.5% sucralose;
2.0% ZEODENT 113:
1.0% STEPANOL WA-100;
3.0% Cl EMOPHOR RH-40; and
0.3% CARBOPOL 974P.

### EXAMPLE 8

The eighth example is a prophy paste containing:
62.0% ascorbic acid crystals and or granulars;
2.0% sorbitol powder;
0.5% hydroxyapatite crystals;
0.01% sodium saccharine;
0.2% sodium benzoate;
0.2% spearmint/peppermint flavor;
2.0% zinc citrate trihydrate:
23.3% anhydrous glycerin;
0.24% sodium fluoride;
1.0% calcium orthophates;
2.0% hydrated silica;
5.0% tricalcuim phosphate;
1.0% sodium lauryl sulfate; and
0.5% other ingredients.

### EXAMPLE 9

The ninth example is a prophy paste containing:
22% Water-balance in wax;
22% carboxymethylcellulose;
20% ascorbic acid;
10% vegetable glycerin;
5% hydroxyapatite;
5% sodium silicate;
5% VEEGUM (magnesium aluminum silicate);
5% paraffin wax;
4% monosodium phosphate;
1% flavor;
0.24% sodium fluoride;
0.20% color; and
0.56 other ingredients.

### EXAMPLE 10

The tenth example is a privily paste containing:
17% Water-balance in wax;
22% carboxymethylcellulose;
25% ascorbic acid:
10% vegetable glycerin;
5% hydroxyapatite;
5% sodium silicate;
5% VEEGUM (magnesium aluminum silicate);
5% paraffin wax;
4% monosodium phosphate;
1% flavor;
0.24% sodium fluoride;
0.20% color; and
0.55% other ingredients.

### EXAMPLE 11

The eleventh example is a prophy paste containing:
18% Water-balance in wax;
25% carboxymethylcellulose;
25% ascorbic acid;
11% vegetable glycerin;
0.5% hydroxyapatite;
5% sodium silicate;
5% VEEGUM (magnesium aluminum silicate);
5% paraffin wax;
4% monosodium phosphate;
1% flavor;
0.3% sodium fluoride; and
0.2% color.

Although the embodiments of the present disclosure have been described with specific examples, it is to be understood that the disclosure is not limited to those specific examples and that various other changes, combinations and modifications will be apparent to one of ordinary skill in the art without departing, from the scope and spirit of the invention which is to be determined with reference to the following claims.

1. A plaque and tartar removal and prevention dental prophy paste with enamel strengthening, repairing and protecting qualities for oral hygiene comprising:
at least 20% ascorbic acid for plaque and tartar removal;
a blend of hydroxyapatite crystals and sodium fluoride sufficient to strengthen tooth enamel, protect the tooth, enamel against acid damage and repair fissures in the enamel in which plaque causing bacteria collect, the blend containing between 0.2-0.5% hydroxyapatite crystals and about 0.05-0.5% sodium fluoride; and
an amount of glycerin.

2. The plaque and tartar removal and prevention dental prophy paste of claim 1 wherein combining a sufficient quantity of the plaque and tartar removal and prevention substance with saliva in a mouth forms a liquid that chemically dissolves and detaches plaque from teeth, gum and tongue and quickly breaks a barrier between the teeth and calcified tartar.

3. The plaque and tartar removal and prevention dental prophy paste of claim 1 further comprising:
an amount of tricalcium phosphate to use as a carrier for the ascorbic acid, the amount being between 0.25-2.5% tricalcium phosphate.

4. The plaque and tartar removal and prevention dental prophy paste of claim 3 further comprising:
an amount of hydrated silica sufficient to use as a whitener, the amount being between 0.25-4.0% hydrated silica.

5. The plaque and tartar removal and prevention dental prophy paste of claim 4 further comprising:
an amount of sodium saccharine sufficient to use as a sweeter, the amount being between 0.001-0.2% sodium saccharine.

6. The plaque and tartar removal and prevention dental prophy paste of claim 5 further comprising:
an amount of sodium benzoate, the amount being between 0.05-0.4% sodium benzoate.

7. The plaque and tartar removal and prevention dental prophy paste of claim 6 further comprising:
an amount of spearmint/peppermint flavor, the amount being between 0.05-0.4% spearmint/peppermint flavor.

8. The plaque and tartar removal and prevention dental prophy paste of claim 7 further comprising:
an amount of calcium orthophates, the amount being between 0.25-2.5% calcium orthophates.

9. The plaque and tartar removal and prevention dental prophy paste of claim 8 further comprising:
an amount of tricalcuim phosphate, the amount being between 2.0-7.5% tricalcuim phosphate.

10. The plaque and tartar removal and prevention dental prophy paste of claim 9 further comprising:
an amount of sodium lauryl sulfate, the amount being between 0.25-2.5% sodium lauryl sulfate.

11. A plaque and tartar removal and prevention dental prophy paste with enamel strengthening, repairing and protecting qualities for oral hygiene comprising:
at least 20% ascorbic acid for plaque and tartar removal; and
a blend of hydroxyapatite crystals and sodium fluoride sufficient to strengthen tooth enamel, protect the tooth enamel against acid damage and repair fissures in the enamel in which plaque causing bacteria collect, the blend containing between 0.2-0.5% hydroxyapatite crystals and about 0.05-0.5% sodium fluoride.

12. The plaque and tartar removal and prevention dental prophy paste of claim 11 further comprising:
an amount of zinc citrate trihydrate sufficient to prevent plaque adherence to the tooth enamel.

13. The plaque and tartar removal and prevention dental prophy paste of claim 12 further comprising:
an amount of anhydrous glycerin.

14. The plaque and tartar removal and prevention dental prophy paste of claim 11 wherein combining a sufficient quantity of the plaque and tartar removal and prevention substance with saliva in a mouth forms a liquid that chemically dissolves and detaches plaque from teeth, gum and tongue and quickly breaks a barrier between the teeth and calcified tartar.
